(19) United States
(12) Patent Application Publication
(10) Pub. No.: US 2008/0124289 A1
(43) Pub. Date: May 29, 2008

(54) Use of an alkyloxazolidinone as a moisturizing cosmetic agent and method of moisturizing the skin

(75) Inventors: Chantal Kurfurst, Saint Jean De Braye (FR); Beatrice Beaufrere, Olivet (FR); Brigitte Noe, Orleans (FR)

Correspondence Address: HAMRE, SCHUMANN, MUELLER & LARSON, P.C., P.O. BOX 2902, MINNEAPOLIS, MN, 55402-0902, US

(73) Assignee: LVMH RECHERCHE, Saint Jean De Braye (FR)

(21) Appl. No.: 11/982,570

(22) Filed: Nov. 2, 2007

(30) Foreign Application Priority Data
Nov. 16, 2006 (FR) 0654933

(51) Int. Cl. A61K 8/49

(52) U.S. Cl. 424/70.1

(57) ABSTRACT

A 4-alkyloxazolidin-2-one, particularly 4-decyloxazolidin-2-one, is used as a moisturizing cosmetic agent or as a cosmetic active in a method of cosmetic care for moisturizing skin without significant stickiness.

The present invention relates essentially to a novel use of a 4-alkyl-oxazolidin-2-one, particularly 4-decyloxazolidin-2-one, as a cosmetic agent or cosmetic active ingredient for the manufacture of a cosmetic composition with moisturizing activity.

### STATE OF THE ART

4-Alkyloxazolidin-2-one molecules are known to those skilled in the art.

Some have been described especially in the paper by Thomas A. Foglia et al. published in the journal J. Org. Chem., 1967, 32 (1), pages 75-78. Patent document U.S. Pat. No. 4,960,771 also describes the use of 4-decyloxazolidin-2-one as an agent for promoting the transdermal penetration of an active principle contained in a composition for treating humans or animals.

According to patent document WO 9633706, this molecule has also been used in an anesthetic pharmaceutical composition in association with a local anesthetic.

### OBJECTS OF THE INVENTION

One main object of the present invention is to provide a novel cosmetic agent having a moisturizing effect which exhibits a very good biocompatibility and imparts, to the cosmetic composition for topical use in which it is incorporated, moisturizing properties on the corporeal skin, particularly facial skin, to which said composition is applied.

Another main object of the present invention is to provide a novel cosmetic agent or novel cosmetic active ingredient having a moisturizing effect which is simultaneously capable of improving the cosmetic qualities of the compositions in which it is incorporated, particularly their feel and unctuousness, without thereby making the composition sticky when applied.

### SUMMARY OF THE INVENTION

According to a first feature, the present invention relates to the use, as a moisturizing cosmetic agent for the manufacture of a moisturizing cosmetic composition, of a 4-alkyloxazolidin-2-one of the following chemical formula:

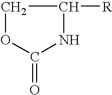

in which the substituent R is selected from a linear or branched alkyl group comprising from 4 to 24 carbon atoms, advantageously from 4 to 18 carbon atoms; or from a linear or branched alkylene group comprising from 4 to 24 carbon atoms, advantageously from 4 to 18 carbon atoms.

Throughout the specification and claims, an alkylene group means an alkylene having at least one carbon-carbon unsaturated bond, in particular 1, 2 or 3 unsaturated bonds.

It has in fact been discovered, surprisingly, that the 4-alkyloxazolidin-2-ones of the invention have a pronounced moisturizing effect. Furthermore, when they are incorporated into a cosmetic composition, they give this composition effective moisturizing properties together with other particularly valuable cosmetic properties, such as a remarkable feel and unctuousness, which are highly sought-after in cosmetics, without thereby giving rise to stickiness.

In other words, these substances possess a remarkable moisturizing power without having the disadvantages usually inherent in the majority of fatty substances.

In one preferred embodiment of the invention, said 4-alkyloxazolidin-2-one is 4-decyloxazolidin-2-one, where said substituent R is a linear decyl radical.

In another advantageous embodiment of the invention, this substance is incorporated in a concentration of between 0.05% and 10%, preferably of between 0.5% and 10% and particularly preferably of between 1 and 5% by weight of the final cosmetic composition. “Final cosmetic composition” is understood as meaning the cosmetic composition ready for use.

Of course, this substance can be associated with various other cosmetically acceptable active principles that are incorporated for the manufacture of cosmetic compositions intended for skin care.

In another advantageous embodiment of the invention, if said cosmetic composition comprises a fatty phase, particularly if it is in the form of an emulsion, the 4-alkyloxazolidin-2-one substance, particularly 4-decyloxazolidin-2-one, is incorporated into said fatty phase.

It is also advantageously possible to incorporate, into said cosmetic composition, effective amounts of other cosmetic agents or active principles for improving the moisturizing character of the composition, such as glycerol and urea, or agents for promoting keratinocyte differentiation, such as ecdysteroids, especially ecdysterone and turkesterone, or plant extract containing them, such as extracts of Cyanotis arachnoidea or Ajuga turkestanica. It is further possible to add other cosmetic active agents such as an alpha-hydroxy acid, especially C2-C12, such as citric acid, glycolic acid, gluconic acid, malic acid, lactic acid and 2-hydroxybutyric acid, vitamin A, preferably in the form of an ester, pyrrolidonecarboxylic acid, a sugar such as xylose, vitamin B5 and an extract of white lupin (Lupinus albus).

Furthermore, as can also be easily understood by those skilled in the art, it is possible to add any cosmetically acceptable excipient for the manufacture of said cosmetic composition, particularly, of course, cosmetically acceptable excipients or agents which make it possible to manufacture a fatty phase and to manufacture an aqueous phase and a stable emulsion. Moreover, it is of course possible to add various preservatives, fragrances, etc.

According to a second feature, the present invention further relates to a method of cosmetic care for obtaining a moisturizing effect on the skin, which comprises selecting at least one area of skin in need of hydration, and applying to the areas of skin in need of such hydration, as a moisturizing cosmetic agent, a moisturizing effective amount of a 4-alkyloxazolidin-2-one of the following chemical formula:

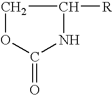

in which the substituent R is selected from a linear or branched alkyl group comprising from 4 to 24 carbon atoms, advantageously from 4 to 18 carbon atoms; or from a linear or branched alkylene group comprising from 4 to 24 carbon atoms, advantageously from 4 to 18 carbon atoms.

By the terms “a moisturizing effect amount”, it is meant an amount effective to provide a moisturizing effect on the skin. One skilled in the art very well knows how to observe or to determine such a moisturizing effect.

Those skilled in the art are perfectly capable of selecting the areas of skin in need of hydration. They are generally the areas of corporeal skin that are most exposed to solar radiation or most sensitive to the environment (climatic changes, low humidity, rubbing, etc.), mainly the face and the upper and lower limbs.

In one particular variant, this cosmetic care will be cosmetic care for moisturizing the skin.

The particular embodiments of the invention described above can obviously be applied to the method of cosmetic care in a similar manner.

Other objects, characteristics and advantages of the invention will become clearly apparent from the following explanatory description referring to a currently preferred embodiment of the invention, which is given simply by way of illustration and cannot therefore limit the scope of the invention in any way. In the Examples the percentages are given by weight, the temperature is room temperature or is given in degrees Celsius and the pressure is atmospheric pressure, unless indicated otherwise.

Example 1 below forms an integral part of the invention, and any characteristic that appears novel relative to a state of the art is claimed as such and as a general means.

### EXAMPLE 1 ACCORDING TO THE INVENTION

### Cosmetic Composition in the Form of a Cream with a Moisturizing Effect According to the Invention

The cosmetic composition in the form of a cream with a moisturizing effect is prepared from the following ingredients, given in percentages by weight:

1°) Oily phase A

| | |
|---|---|
| a) Hydrogenated polyisobutene | 14.5 |
| b) Caprylic triglycerides | 6.0 |
| c) Cetearyl ethylhexanoate | 3.9 |
| d) Steareth-21 | 2.2 |
| e) Cetyl palmitate | 1.6 |
| f) Glyceryl stearate | 1.5 |
| g) Stearic acid | 1.4 |
| h) 95% cetyl alcohol | 1.0 |
| i) 98% stearyl alcohol | 1.0 |
| j) Steareth-2 | 0.8 |
| k) 4-Decyloxazolidin-2-one | 2.0 |

2°) Aqueous phase B

| | |
|---|---|
| a) Phenoxyethanol (or phenoxetol) | 0.70 |
| b) Carbomer (Carbopol 940 ®) | 0.20 |
| c) Purified water | 62.40 |

3°) Neutralizing aqueous phase C

| | |
|---|---|
| 10% by weight sodium hydroxide solution | 0.65 |

4°) Fragrance phase D

| | |
|---|---|
| Fragrance concentrate | 0.15 |
| TOTAL | 100.00 |

This cosmetic composition in the form of an emulsion is prepared in the following manner:

a) Firstly the aqueous phase B is prepared by initially mixing the phenoxetol with the purified water, heating to 85° C. and then cooling to 25° C., and then adding the gelling agent, such as a carbomer (like Carbopol 940°), with stirring.

This aqueous phase B is heated again to 85° C.

b) The oily phase A is prepared separately by mixing all the ingredients of the oily phase listed above under 1°), except for the cosmetically active agent 4-decyloxazolidin-2-one. This mixture is heated up to 85° C. to give a homogeneous oily phase, and then adding the cosmetically active agent according to the invention, 4-decyloxazolidin-2-one.

c) The aqueous phase B is then added to the oily phase A, with stirring, these two phases being at a temperature of about 85° C.

d) This mixture is left to cool for a few minutes and, when the temperature has dropped to about 70° C., the neutralizing aqueous phase C, consisting of the 10% sodium hydroxide solution, is added.

e) The emulsion obtained is left to cool further and, when the temperature has dropped to about 35° C., the fragrance concentrate of phase D is added under stirring.

The cosmetic composition obtained in this way can be marketed as such. It is hence used to measure the moisturizing effect as described in Example 3.

### COMPARATIVE EXAMPLE 2

The same ingredients and the same phases are used as those described in the preparation of the composition of Example 1 according to the invention, except that the cosmetically active agent according to the invention, 4-decyloxazolidin-2-one, is not introduced, so it is in a proportion of 0% here, and its absence is compensated by the addition of a further 2% of hydrogenated polyisobutene, which is thus used here to make up the balance and is then in a concentration of 16.5% instead of the 14.5% in Example 1.

The preparation of this composition is thus identical to that of Example 1 except that there is no special step for addition of the active principle, which is not present in this comparative composition.

### EXAMPLE 3

### Test for Measuring the Moisturizing Effect of the Active Principle of Example 1 According to the Invention, 4-decyloxazolidin-2-one, Relative to the Comparative Composition of Example 2

The moisturizing power of the active substance according to the invention, 4-decyloxazolidin-2-one, was evaluated by corneometry, the composition of Example 1 according to the invention, in which it is incorporated, being compared with the comparative or ‘control’ composition of Example 2, in which it is not incorporated.

The corneometer is an apparatus for measuring the variation in capacitance associated with a variation in the water content of the skin.

An electric field is created on the surface of the skin by means of a probe made up of two electrodes. The capacitance of the electrode/skin system is influenced by changes in the dielectric constant of the biological medium in contact with the probe. The greater the hydration of the skin, the higher is the dielectric constant. The variations in capacitance are expressed in arbitrary units.

The measurement of electrical properties is recognized as being an objective method of measuring the change in skin hydration, as described in the paper by E. Berardesca in the name of the European Group for Efficacy Measurements on Cosmetics and Other Topical Products (EEMCO), published in the journal Skin Research and Technology, 1997, vol. 3, pages 126-132, under the title “EEMCO Guidance for the Assessment of Stratum Corneum Hydration: Electrical Methods”.

Three areas of skin 25 centimeters square were defined on the inner forearm of 6 healthy volunteers. The composition according to the invention and the control comparative composition were each applied to one of the three defined areas at a rate of 2 mg/cm², the distribution of said areas being random; the third area is untreated and serves as a control. The hydration level of the skin was measured by means of a corneometer marketed by the German company Courage+Khazaka (Germany) under the reference CM820. The measurement is made before and 2 and 6 hours after application of the two compositions. The percentage variations in hydration are expressed relative to the untreated control area prior to application.

| Composition | After 2 hours | After 6 hours |
|---|---|---|
| Example 1 - invention | +22.3% | +16.6% |
| Example 2 - comparative | +17.7% | +11.4% |

Statistical analysis shows that the overall difference observed between the two compositions is significant (p=0.02).

It is seen from these results that the compositions containing the cosmetic active agent according to the invention, 4-alkyloxazolidin-2-one, has a significant moisturizing effect relative to the comparative composition. The composition according to the invention can thus be applied to the areas of skin that have previously been selected as being in need of hydration, as described above, thereby carrying out the method of cosmetic care described above.

What is claimed is:

1. A method of cosmetic care for obtaining a moisturizing effect on the skin, which comprises selecting at least one area of skin in need of hydration, and applying to said area of skin in need of such hydration, as a moisturizing cosmetic agent, a moisturizing effective amount of a 4-alkyloxazolidin-2-one of the following chemical formula: 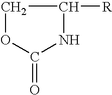
in which the substituent R is selected from a linear or branched alkyl group comprising from 4 to 24 carbon atoms, advantageously from 4 to 18 carbon atoms; or from a linear or branched alkylene group comprising from 4 to 24 carbon atoms, advantageously from 4 to 18 carbon atoms.

2. The method of claim 1, wherein said 4-alkyloxazolidin-2-one is 4-decyloxazolidin-2-one, where said substituent R is a linear decyl radical.

3. The method of claim 1, wherein said 4-alkyloxazolidin-2-one is incorporated into the cosmetic composition in a concentration of between 0.05% and 10%.

4. The method of claim 1, wherein said 4-alkyloxazolidin-2-one is incorporated into the cosmetic composition in a concentration of between 0.5% and 10%.

5. The method of claim 1, wherein said 4-alkyloxazolidin-2-one is incorporated into the cosmetic composition in a concentration of between 1 and 5% by weight.

6. The method of claim 1, wherein said cosmetic composition comprises a fatty phase and the 4-alkyloxazolidin-2-one is incorporated into said fatty phase.

7. The method of claim 6, wherein said cosmetic composition comprises 4-decyloxazolidin-2-one incorporated into said fatty phase.

8. The method of claim 1, wherein said cosmetic composition comprises an effective amount of another cosmetic agent.

9. The method of claim 8, wherein said another cosmetic agent is a moisturizing agent improving moisturizing of the skin.

10. The method of claim 9, wherein said moisturizing agent comprises at least one of glycerol and urea.

11. The method of claim 8, wherein said another cosmetic agent is an agent for promoting keratinocyte differentiation.

12. The method of claim 11, wherein said agent for promoting keratinocyte differentiation comprises an ecdysteroid.

13. The method of claim 12, wherein said ecdysteroid is selected from ecdysterone, turkesterone and any mixture thereof.

14. The method of claim 11, wherein said agent for promoting keratinocyte differentiation comprises a plant extract containing an ecdysteroid.

15. The method of claim 14, wherein said plant extract is selected from an extract of Cyanotis arachnoidea, an extract of Ajuga turkestanica and any mixture thereof.
